(19) United States
(12) Patent Application Publication
(10) Pub. No.: US 2006/0233887 A1
(43) Pub. Date: Oct. 19, 2006

(54) Bioactive material for use in stimulating vascularization

(75) Inventor: Richard M. Day, Harrow (GB)

Correspondence Address: EDWARDS & ANGELL, LLP, P.O. BOX 55874, BOSTON, MA, 02205, US

(73) Assignee: The North West London Hospitals N H S Trust

(21) Appl. No.: 10/545,766

(22) PCT Filed: Feb. 13, 2004
(86) PCT No.: PCT/GB04/00578

§ 371 Date: May 22, 2006

(30) Foreign Application Priority Data
Feb. 14, 2003 (GB) 0303371.9
Oct. 10, 2003 (GB) 0323816.9

(51) Int. Cl. A61K 33/42; C03C 3/097

(52) U.S. Cl. 424/602; 501/63

(57) ABSTRACT

The present invention relates to a bioactive material, particularly one which comprises SiO₂ and CaO and optionally Na₂O and/or P₂O₅, for use in stimulating vascularisation and pharmaceutical compositions, wound dressings, tissue constructs and delivery systems which include such a bioactive material.

The invention relates to a support for biological tissue growth and to a method of stimulating vascularization, particularly by stimulating the release of vascular endothelial growth factor (VEGF).

A major limitation of tissue engineering for the replacement of diseased or damaged tissue is the inability to induce rapid vascular in-growth during tissue development (Mooney et al. “Long-term engraftment of hepatocytes transplanted on biodegradable polymer sponges” J. Biomed. Mater. Res. 1997;37:413-420). Neovascularization is critical to the success of the engineered tissue because blood vessels provide growing cells with oxygen and nutrients necessary for survival. Neovascularization of the tissue construct may be enhanced through the controlled delivery of bioactive phases, such as specific angiogenic growth factors, including vascular endothelial growth factor (VEGF), a potent mitogen for human micro and macrovascular endothelial cells (Leung et al. “Vascular endothelial growth factor is a secreted angiogenic mitogen” Science 1989 8;246(4935):1306-9). To enable larger volumes of tissue to be engineered, the problem of how to enhance the viability of native and/or transplanted cells within tissue engineering scaffolds by inducing neovascularization in the scaffolds needs to be solved.

Synthetic polymer scaffolds for tissue engineering have been primarily composed of members of the poly(α-hydroxy acid) family of polymers (also known as aliphatic polyesters or poly(α-hydroxyesters)), such as poly(L-lactic acid) (PLLA), poly(lactic-co-glycolic acid) (PLGA) and poly(glycolic acid) (PGA). These materials are biocompatible, undergo controllable hydrolytic degradation into natural metabolites, and are Food and Drug Administration (FDA) approved for certain clinical applications.

In addition to acting as matrix structures for support of tissue growth, ways in which these biodegradable polymer scaffolds may be developed into delivery devices for growth factors during tissue engineering have been investigated. These have included creating sustained release of growth factors by incorporating the bioactive molecules, such as VEGF, directly into the scaffold at or after fabrication (Murphy et al. “Sustained release of vascular endothelial growth factor from mineralized poly(lactide-co-glycolide) scaffolds for tissue engineering” Biomaterials 2000; 21:2521-2527; Sheridan et al. “Bioabsorbable polymer scaffolds for tissue engineering capable of sustained growth factor delivery” J Control Release 2000 14;64(1-3):91-102). Alternatively, angiogenic growth factors have been delivered into the scaffold via co-transplantation of growth factor-secreting cells that are either natural or genetically engineered, as recently described for hepatocyte growth factor (Hidaka et al. “Formation of vascularized meniscal tissue by combining gene therapy with tissue engineering” Tissue Eng. 2002; 8:93-105). However, fabrication of scaffolds involves the use of organic solvents and/or high temperatures. These conditions are unsuitable for the incorporation of bioactive peptides, such a VEGF.

A way of ameliorating these problems has been sought.

According to the invention there is provided a bioactive material for use in stimulating vascularisation, preferably for use in inducing secretion of an endothelial cell mitogen (especially VEGF).

According to the invention there is also provided use of a bioactive material in the manufacture of a medicament for use in stimulating vascularisation, preferably for use in inducing secretion of an endothelial cell mitogen (especially VEGF).

Bioactive materials are known to the art, and typically contain at least SiO₂ and CaO. The bioactive material preferably optionally comprises:

P₂O₅, CaF₂, MgO, Al₂O₃, TiO₂, phosphate ions, SrO, K₂O, B₂O₃, fluoride ions, Na₂O and/or Ag₂O. More preferably the bioactive material optionally comprises Na₂O and/or P₂O₅.

Examples of a suitable bioactive material include the following (for each example, the ingredients are given in decreasing order of their relative amount):

CaO, SiO₂, P₂O₅, CaF₂

CaO, MgO, SiO₂, P₂O₃, CaF₂

CaO, P₂O₅, SiO₂, Al₂O₃, TiO₂

CaO, P₂O₅, SiO₂, CaF₂

CaO, phosphate ions, SiO₂

CaO, SrO, SiO₂, P₂O₅, CaF₂

K₂O, MgO, Al₂O₃, B₂O₃, SiO₂, CaO, P₂O₅, fluoride ions

K₂O, MgO, CaO, Al₂O₃, B₂O₃, SiO₂, P₂O₅, fluoride ions

MgO, CaO, SiO₂

Na₂O, CaO, MgO, Al₂O₃, SiO₂, P₂O₅, CaF₂

Na₂O, CaO, P₂O₅, SiO₂

Na₂O, K₂O, MgO, CaO, B₂O₃, P₂O₅, SiO₂

SiO₂, Na₂O, CaO, P₂O₅, K₂O, Al₂O₃, MgO

SiO₂, Al₂O₃, P₂O₅, Na₂O, K₂O, CaO, fluoride ions

SiO₂, CaO

SiO₂, CaO, Na₂O, P₂O₅

SiO₂, CaO, P₂O₅

SiO₂, CaO, P₂O₅, Ag₂O

SiO₂, MgO, Al₂O₃, K₂O, CaO, P₂O₅, fluoride ions

SiO₂, Na₂O, CaO, P₂O₅, Al₂O₃, B₂O₃

SiO₂, Na₂O, K₂O, CaO, MgO, P₂O₅

TiO₂, SiO₂, CaO, B₂O₃

A bioactive material preferably contains from 45 to 90% o of SiO₂ (typically less than 60 mol. %) and from 10 to 55% of CaO and optionally Na₂O, N₂O₅ and/or P₂O₅, especially a high sodium oxide and CaO content (20-25% each); wherein the percentages are by weight or are molar percentages; preferably the percentages are by weight. There is preferably a high molar ratio of calcium to phosphorus (from 4:1 to 6:1, preferably about 5:1). Examples of suitable bioactive materials contain 60 mol. % SiO₂, 40 mol. % CaO; 70 mol. % SiO₂, 30 mol. % CaO; 60 mol. % SiO₂, 36 mol. % CaO, 4 mol. % P₂O₅; 80 mol. % SiO₂, 16 mol. % CaO, 4 mol. % P₂O₅; 46.1 mol. % SiO₂, 24.4 mol. % Na₂O, 26.9 mol. % CaO, 2.6 mol. % P₂O₅. The bioactive material is preferably a bioactive ceramic, gel-glass or glass material. The advantage of a bioactive gel-glass material is that it can contain a greater amount of SiO₂. More preferably the bioactive material is a bioactive ceramic material, especially a bioactive ceramic material sold under the brandname Bioglass®, more especially 45S5 Bioglass®.

The amount of bioactive material generally used is preferably from 0.00001 wt %, more preferably from 0.001 wt %, most preferably from 0.01 wt % to 10 wt %, more preferably to 5 wt %, particularly preferably to 2 wt %, most preferably to 1 wt %. Where the bioactive material is used in the form of a coating, the amount of bioactive material used is preferably from 0.00003125 mg/cm² (0.00001 wt %), more preferably from 0.003125 mg/cm² (0.001 wt %), most preferably from 0.03125 mg/cm² (0.01 wt %) to 6.25 mg/cm² (2 wt %), preferably to 3.125 mg/cm² (1 wt %), most preferably to 1.5625 mg/cm² (0.5 wt %).

Such materials are called “bioactive” because when they are implanted into a human or animal body, interfacial bonds form between the material and surrounding tissues. When such glasses are exposed to water or body fluids, several key reactions occur. The first is cation exchange wherein interstitial sodium and calcium ions from the glass are replaced by protons from solution, forming surface silanol groups and nonstoichiometric hydrogen-bonded complexes. This cation exchange increases the hydroxyl concentration of the solution, leading to attack of the fully dense silica glass network to produce additional silanol groups and controlled interfacial dissolution. As the interfacial pH becomes more alkaline and the concentration of hydrolyzed surface silanol groups increases, the conformational dynamics attending high numbers of proximal silanol groups, combined with the absence of intestitial ions, cause these groups to repolymerize into a silica-rich surface layer. Another consequence of alkaline pH at the glass-solution interface is crystallization into a mixed hydroxyapatite phase of the CaO and P₂O₅ that were released into solution during the network dissolution. This takes place on the SiO₂ surface. This phase contains apatite crystallites which nucleate and interact with interfacial components such as glycosaminoglycans, collagen and glycoproteins. It is thought that incorporation of organic biological constituents within the growing hydroxyapatite- and SiO₂-rich layers triggers close interactions with living tissues characteristic of bioactivity. See Greenspan et al. (1994), Bioceramics 7:55-60.

In stating that the bioactive material induces vascularisation, we mean that the bioactive material is useful in therapeutic angiogenesis. The bioactive material according to the invention is particularly useful in the treatment of cardiovascular disease such as ischaemic heart disease, peripheral vascular disease, and/or congestive heart failure; non-cardiovascular disease such as renovascular, cerebrovascular and/or peptic ulcer; wound-healing; tissue-engineering; tissue regeneration such as in the treatment of a burn, plastic and/or reconstructive surgery (e.g. skin-grafts), and/or chronic wound healing (e.g. diabetic ulcers).

It has now been surprisingly found that a bioactive material induces secretion of the endothelial cell mitogen called vascular endothelial growth factor, by fibroblasts. The ability of a bioactive material, particularly 45S5 Bioglass®, to stimulate the release of VEGF from transplanted and/or host fibroblasts that have migrated into tissue engineering scaffolds containing 45S5 Bioglass' will be extremely beneficial, as the goal in tissue engineering is to induce rapid vascular in-growth sufficient to meet the metabolic requirements of the engineered tissue.

Whilst the Example 1 of the present application shows the ability of a bioactive material (45S5 Bioglass®) to induce secretion of VEGF, it is to be expected that other endothelial cell mitogens such as acidic or basic fibroblast growth factors (aFGF and bFGF respectively), angiogenin (ANG), hepatocyte growth factor (HGF), epidermal growth factor (EGF), angiopoietins and platelet-derived endothelial cell growth factor (PDGF) will respond to Bioglass® in a similar manner.

The bioactive material may optionally be used in association with a further therapeutic agent such as an antibiotic, antiviral, healing promotion agent, anti-inflammatory agent, immunosuppressant, growth factor, antimetabolite, cell adhesion molecule (CAM), bone morphogenic protein (BMP), vascularizing agent, anti-coagulant, and/or topical anesthetic/analgesic.

A preferable antibiotic is a topical antibiotic suitable for skin treatment. Examples of such antibiotics include but are not limited to: chloramphenicol, chlortetracycline, clyndamycin, clioquinol, erythromycin, framycetin, gramicidin, fusidic acid, gentamicin, mafenide, mupiroicin, neomycin, polymyxin B, bacitracin, silver sulfadiazine, tetracycline and/or chlortetracycline.

A suitable anti-viral includes a topical anti-viral, such as acyclovir andor gancyclovir. A suitable anti-inflammatory agent includes a corticosteroid, hydrocortisone and/or a non-steroidal anti-inflammatory drug. A suitable growth factor includes a basic fibroblast growth factor (bFGF), epithelial growth factor (EGF), transforming growth factors alpha and/or beta (TGF alpha and/or beta), platelet-derived growth factor (PDGF), and/or vascular endothelial growth factor/vascular permeability factor (VEGF/VPF)). A suitable topical anaesthetic includes benzocaine and/or lidocaine.

The invention can be applied in a number of different ways. Examples include the use of the invention to aid wound healing through stimulation of angiogenesis; use of the invention in a new tissue construct, e.g. with a biodegradable polymer to aid new tissue growth or with an artificial membrane to induce vascularization; and use of the invention to create a new tissue construct in vitro which could be used for example in a screen for a new pharmaceutical compound. Accordingly the invention provides the following new compositions of matter which enable the invention to be applied in these different ways.

Wound Healing

It is known that in an aqueous environment a bioactive material will release Si, Ca, P and Na into the local milieu. The changes in elementary composition of the solution are due to ion exchange mechanisms occurring at the glass-solution interface (Hench & West (1996) Biological applications of bioactive glasses. Life Chemistry Reports 13:187-241). Without intending to be limited to any particular theory about how the invention works, it is believed that the soluble ions released are factors that influence the secretion of VEGF by fibroblasts. Indeed aqueous solutions of bioactive glass have been shown to have broad antimicrobial effects (Stoor et al. (1998) Antimicrobial effects of a bioactive glass paste on oral microorganisms. Acta Odontol Scand 56:161-165). Accordingly from the data in Example 1 of the present application, it is expected that a similar paste or ointment will be effective in stimulating secretion of VEGF in wounds.

According to the invention there is provided a pharmaceutical formulation comprising a bioactive material and a pharmaceutically acceptable adjuvant or carrier. The formulation is preferably for use in stimulating vascularization particularly in a wound or a burn; more preferably for use in inducing secretion of an endothelial cell mitogen (especially VEGF) particularly in a wound or a burn.

According to the invention there is also provided use of a bioactive material in the manufacture of a pharmaceutical formulation as defined above for use in stimulating vascularisation, preferably for use in inducing secretion of an endothelial cell mitogen (especially VEGF).

The amount of tissue lost with skin wounds determines whether the edges of the skin can be brought together and secured with a ligature. Wounds left open to heal are dependent on the action of growth factors, oxygen and nutrients. The wound becomes packed with granulation tissue that is rich in blood vessels, a process termed proliferation, which facilitates wound healing (Trudgian “Transorbent hydrocellular wound dressing from Maersk Medical” Br. J. Nurs. 2000;9:2181-2186). The pharmaceutical formulation according to the invention is useful because application of a bioactive material to a wound may enhance the proliferation process by inducing VEGF secretion that would cause the wound to heal faster by promoting angiogenesis.

It is noted that Example 1 of the present application shows that the bioactive material initially induces fibroblast proliferation but then has an inhibitory effect in vitro. The reasons for this are not known but may be due to cell differentiation. This effect may not be observed in vivo where the environment has a better buffering capacity. Even if inhibition does occur in vivo, it is to be expected that this may, in fact, be of benefit because a reduction in fibroblast infiltration/proliferation in response to a bioactive material might enable other cell types (e.g. endothelial cells) to migrate into the granulation tissue of wounds or a tissue construct that might otherwise be overwhelmed by fibroblasts.

The formulation may be suitable for oral, rectal, topical or parenteral (including subcutaneous, intramuscular and intravenous) administration. The formulation according to the invention is preferably designed for topical administration to a wound or a burn. Typical of such formulations are ointments, creams, and gels. A preferred formulation for use in accordance with this invention is an ointment.

Ointments generally are prepared using either (1) an oleaginous base, i.e., one consisting of fixed oils or hydrocarbons, such as white petrolatum or mineral oil, or (2) an absorbant base, i.e., one consisting of an anhydrous substance or substances which can absorb water, for example, anhydrous lanolin. Customarily, following formation of the base, whether oleaginous or absorbent, the bioactive material may be added in an amount affording the desired concentration.

Creams are generally oil/water emulsions. They consist of an oil phase (internal phase), comprising typically fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate; hydrophilic colloids, such as acacia colloidal clays, veegum, and the like. Upon formation of the emulsion, the bioactive material may be added in an amount to achieve the desired concentration.

Gels comprise a base selected from an oleaginous base, water, or an emulsion-suspension base, such as described above. To the base is added a gelling agent which forms a matrix in the base, increasing its viscosity. Examples of gelling agents are hydroxypropyl cellulose, an acrylic acid polymer, glyceryl monooleate, and the like. The bioactive material may be added to the formulation at the desired concentration at a point preceding addition of the gelling agent.

To provide the maximum available surface area, the bioactive material is preferably included in the pharmaceutical formulation in the form of a powder. The amount of bioactive material used in the pharmaceutical formulation according to the invention is preferably from 0.00001 wt %, more preferably from 0.001 wt %, most preferably from 0.01 wt % to 10 wt %, more preferably to 5 wt %, particularly preferably to 2 wt %, most preferably to 1 wt %.

According to the invention there is further provided a wound dressing including a dressing layer having an upper surface and a wound facing surface and a layer of bioactive material wherein the layer of bioactive material is applied to the wound facing surface of the dressing layer.

For the purposes of this specification, the term “dressing” includes bandages, i.e. in which the wound-contacting part of the system is part of a larger product.

The layer of bioactive material is optionally either continuous or discontinuous; preferably it is continuous. The layer of bioactive material preferably includes a biodegradable polymer; more preferably the layer is in the form of a bioactive material/biodegradable polymer composite or foam.

The dressing layer may optionally take any form generally known in the art. A full description of dressings and wound management, including the various types of dressing layer that may be used in this invention, may be found in “A Prescriber's Guide to Dressings and Wound Management Materials” (March 1996) National Health Service, Wales; the content of this document is incorporated herein by reference.

The wound dressing may optionally be in the form of a traditional dressings (gauze, cotton wool, lint, gamgee etc.), a low-adherent dressing, a vapour permeable film/membrane, a hydrogel, a hydrocolloid, a polysaccharide dressing, an alginate, a foam, a de-odoriser, a paste bandage, tulles (plain or medicated), and/or an anti-microbial dressing.

The wound dressing according to the invention is preferably in the form of a dressing which has been developed with the aim of supporting proliferation, such as a polyurethane foam dressing or a hydrocolloid dressing, for example, one manufactured from pectin, gelatine, a hydrophobic polymer and/or carboxymethylcellulose (Trudgian “Transorbent hydrocellular wound dressing from Maersk Medical” Br. J. Nurs. 2000;9:2181-2186).

The dressing according to the invention may optionally include a further layer such as an adhesive layer and/or a removable protective layer.

According to the invention there is also provided a method of inducing vascularization in a wound or burn which method comprises applying to a patient in need of such treatment an effective amount of a bioactive material.

In the method of inducing vascularization according to the invention, the bioactive material is preferably in the form of a pharmaceutical formulation or a wound dressing according to the invention.

According to the invention there is also provided a ligature including linking means for joining a first side and a second side of a wound together wherein the means is coated and/or impregnated with a bioactive material. The ligature according to the invention is optionally in the form of a suture, surgical staple or adhesive strip. A ligature according to the invention preferably contains a surface coating of bioactive material. The amount of bioactive material used in the coating is preferably from 0.00003125 mg/cm² (0.00001 wt %), more preferably from 0.003125 mg/cm² (0.001 wt %), most preferably from 0.03125 mg/cm² (0.01 wt %) to 6.25 mg/cm² (2 wt %), preferably to 3.125 mg/cm² (1 wt %), most preferably to 1.5625 mg/cm² (0.5 wt %). The bioactive material could be applied to the ligature as either a surface coating or the ligature could be manufactured from a composite of a polymer and the bioactive material. Examples of suitable materials for use as the linking means where the ligature according to the invention is a suture include monofilament, multifilament or braided materials and can be composed of absorbable material (e.g. cat gut, chromic cat gut, polyglycolic acid (Dexon), polyglactic acid (Vicryl), polydioxanone (PDS)) or non-absorbable material (silk, cotton, polyester (Mersilene), Teflon (Tevdek), silicone (Tri-Cron), polybutilate (Ethibond), nylon, polypropylene, stainless steel). Where the ligature is in the form of a surgical staple, the linking means is preferably formed from metal e.g. titanium or stainless steel. The bioactive material may be provided as a coating on the metal or it may be used in the form of a composite with the metal. Where the ligature is in the form of an adhesive strip, it preferably comprises a dressing layer coated with a biocompatible adhesive.

Tissue Constructs

According to the invention there is provided a first tissue construct which consists essentially of a biocompatible material (preferably a biocompatible polymer), a bioactive material and, optionally, one or more biological cells.

The bioactive material used in the invention generally is not affected during synthesis of the construct. It preferably has a smaller size average particle than a bioactive material normally used in implants for promoting new bone or bone connective tissue growth. This is because in a bone implant, the bioactive material performs a different function. The function of the bioactive material in a bone implant is structural, acting as a bonding interface between new bone and the old. In the present invention, the bioactive material functions differently. It has been found that a smaller particle size is better because there is then a greater surface area of bioactive material available to interact. Furthermore, where the biodegradable polymer is porous, a smaller particle size means that the bioactive material does not block the pores of the polymer. Accordingly, the bioactive material is more preferably in the form of a powder. Most preferably it has an average particle size of less than about 5 μm.

According to the invention there is provided a second tissue construct which comprises a porous biocompatible material (preferably a biocompatible polymer) and a particulate bioactive material having an average particle size less than the average size of the pores of the polymer.

The amount of the bioactive material used in the construct according to the invention is preferably less than the amount generally used in implants for promoting new bone or bone connective tissue growth. Such implants use from 10 to 70 wt % of a bioactive material. The amount of bioactive material used in the construct according to the invention is more preferably less than 1 wt %, preferably from 0.001 (more preferably from 0.01) to 1 wt %.

According to the invention there is provided a third tissue construct which comprises a biocompatible material (preferably a biocompatible polymer), a bioactive material in an amount between 0.001 and 10 wt % and, optionally, one or more biological cells.

According to the invention there is also provided a tissue construct according to the first, second and/or third aspect(s) of the invention for use in stimulating new tissue growth, preferably for use in stimulating vascularization, more preferably for use in inducing secretion of an endothelial cell mitogen (especially VEGF).

According to the invention there is further provided use of a biodegradable tissue construct according to the first, second and/or third aspect(s) of the invention in the manufacture of a medicament for use in stimulating new tissue growth, preferably for use in stimulating vascularization, more preferably for use in inducing secretion of an endothelial cell mitogen (especially VEGF).

According to the invention there is provided a method of stimulating new tissue growth which method includes the step of implanting in a patient in need of such treatment a tissue construct according to the first, second and/or third aspect(s) of the invention. Preferably the method of the invention is a method of stimulating vascularization; more preferably it is a method of inducing secretion of an endothelial cell mitogen (especially VEGF).

The biocompatible material used in the constructs according to the invention is preferably biodegradable, preferably such that the construct itself is biodegradable. The advantages of having a biodegradable construct include that over a period of time the quantity of foreign material in the body of a patient reduces as the tissue re-grows such that eventually the implant is replaced by new tissue without any polymer remaining in the body. The term “biodegradable” means capable of breaking down over time inside a patient's body or when used with cells to grow tissue outside of the body. A therapeutic construct is a device used for placement in a tissue defect in a patient (human or animal) to encourage ingrowth of tissue and healing of the defect.

A biocompatible polymer known to the art for producing a biodegradable implant material may be used in this invention as a biocompatible material. Examples of such polymers include polyglycolide (PGA), a copolymer of glycolide such as a glycolide/L-lactide copolymer (PGA/PLLA), a glycolide/trimethylene carbonate copolymer such as poly-L-lactide (PLLA), Poly-DL-lactide (PDLLA), a L-lactide/DL-lactide copolymer; a copolymer of PLA such as a lactide/tetramethylglycolide copolymer, lactide/trimethylene carbonate copolymer, lactide/δ-valerolactone copolymer, lactide ε-caprolactone copolymer, a polydepsipeptide, a PLA/polyethylene oxide copolymer, an unsymmetrically 3,6-substituted poly-1,4-dioxane-2,5-dione; poly-β-hydroxybutyrate (PHBA), a PHBA/β-hydroxyvalerate copolymer (PHBA/HVA), poly-β-hydroxypropionate (PHPA), poly-p-dioxanone (PDS), poly-δ-valerolatone, poly-ε-caprolactone (PCL), a methylmethacrylate-N-vinyl pyrrolidone copolymer, a polyesteramide, a polyester of oxalic acid, a polydihydropyran, a polyalkyl-2-cyanoacrylate, a polyurethane (PU), polyvinyl alcohol (PVA), a polypeptide, poly-β-maleic acid (PMLA), and/or a poly-β-alkanoic acid. The biocompatible material may optionally be a blend of one or more of the above polymers either with or without a natural polymer such as collagen, fibronectin, hyaluronic acid and/or glycosaminoglycan.

The different types of biocompatible polymer differ in the rate at which they biodegrade. In use a balance needs to be struck between selecting a polymer or a combination of polymers which has a sufficiently slow biodegradation rate that there is sufficient time for the construct to support new tissue growth until that tissue is strong enough to support itself whilst not being so slow that the polymer(s) remain for too long before degrading. A skilled person would be able to select a polymer or combination of polymers to give a degradation time suitable for the application for which the construct is designed.

Preferred biodegradable polymers for use as the biocompatible material used in this invention are known to the art, including aliphatic polyesters, preferably a polymer of polylactic acid (PLA), polyglycolic acid (PGA) and a mixture and copolymer thereof, more preferably a 50:50 to 85:15 copolymer of D,L-PLA/PGA, most preferably a 55/45 to 75:25 D,L-PLA/PGA copolymer. A single enantiomer of PLA may also be used, preferably L-PLA, either alone or in combination with PGA.

Preferably the polymeric construct material has a molecular weight of from 25,000 to 1,000,000 Daltons, more preferably from 40,000 to 400,000 Daltons, and most preferably from 55,000 to 200,000 Daltons.

Alternatively the biocompatible material used in the constructs according to the invention is a non-biodegradable biocompatible material. Such a construct is useful to form an artificial membrane in the body, particularly to replace a membrane which normally includes a vascular network such as a tympanic membrane. A suitable non-biodegradable polymer for use in the invention as the biocompatible material is, for example, a microporous PTFE or bisphenol-A poly(carbonate). Alternatively the biocompatible material could be a metal or a metal alloy such as titanium, titanium alloy (e.g. Ti-6AL-4V), stainless steel, cobalt-chromium alloy (e.g. HS25, F-75) and tantalum (Ta). Such a material is particularly useful in inguinal hernia repair in circumstances where routine biodegradable mesh implants do not provide sufficient strength to reinforce the abdominal wall. The bioactive material may be provided as a coating on the metal or it may be used in the form of a composite with the metal.

The constructs according to the invention optionally either comprise a biocompatible polymer coated with a bioactive material or a composite of a biocompatible polymer and a bioactive material. Such a composite may be made by preparing a biocompatible polymer in uncured form, mixing a bioactive material into the polymer, and curing the mixture under conditions of heat and/or pressure to produce a composite construct material.

A tissue construct according to the invention which is composed of a biocompatible polymer/bioactive material composite can also be prepared by a thermally induced phase separation process of polymer solutions and subsequent solvent sublimation, as previously described (“Preparation, characterization, and in vitro degradation of bioresorbable and bioactive composites based on Bioglass®-filled polylactide foams” V. Maquet, A. R. Boccaccini, L. Pravata, I. Notingher, R. Jéröme. J Biomed Mater Res 66A: 335-346, 2003).

In this process, a biocompatible polymer is dissolved in a suitable solvent (e.g. dimethylcarbonate) to produce a polymer weight to solvent volume ratio of 5% (w/v). The mixture is stirred overnight to obtain a homogeneous polymer solution. A suitable amount of bioactive material is added into the polymer solution. The polymer/bioactive material mixture is transferred into a lyophilization flask and sonicated for 15 min to improve the dispersion of the 45S5 Bioglass® into the polymer solution. The flask is then immersed into liquid nitrogen and maintained at −196° C. for 2 h. The frozen mixture is then transferred into an ethylene glycol bath at −10° C. and connected to a vacuum pump (10⁻² Torr). The solvent is sublimed at −10° C. for 48 h and then at 0° C. for 48 h. The sample is completely dried at room temperature in a vacuum oven until reaching a constant weight.

A tissue construct according to the invention which is composed of a porous biocompatible polymer membrane in association with a bioactive material can be prepared by a solvent-casting particulate-leaching technique, as previously described (Preparation and characterization of poly(1-lactic acid) foams. Mikos A, Thorsen A, Czerwonka L, Bao Y, Langer R, Winslow D, Vacanti J. Polymer 1994; 35:1068-77). Such a tissue construct can be prepared by dispersing salt and an optimal amount of a bioactive material in a biocompatible polymer solution. The solvent in which the polymer is dissolved is evaporated to produce a polymer/salt/bioactive material composite. The polymer can then be heated and cooled at a predetermined rate to provide the desired amount of crystallinity. Salt particles are leached out of the membrane by immersing the membrane in water or another solvent for the salt but not the polymer. The membrane is dried, resulting in a porous, biocompatible membrane that contains optimal amounts of a bioactive material.

An example of such a preparation process is to dissolve PLGA in choloroform to yield a 15% w/v polymer solution. Sieved NaCl particles at 100 μm or 300 μm were mixed to the polymer solution; the mixture was cast and the solvent evaporated. Then the resulting solid mixture was used to fill the holes of a steel mold. The samples were compression molded at high temperature and pressure for a suitable time. The samples were removed from the mold and washed with water to leach out the salt.

A tissue construct in the form of a porous, biocompatible membrane that contains an optimal amount of bioactive material can be used to manufacture a bioactive scaffold for use in tissue regeneration. An example of this is a device that will fit snugly into the tract of enterocutaneous or perianal fistula. The device can be manufactured using an extrusion process as described elsewhere (Widmer M S, Gupta P K, Lu L, Meszlenyi R K, Evans G R, Brandt K, Savel T, Gurlek A, Patrick C W Jr, Mikos A G. Manufacture of porous biodegradable polymer conduits by an extrusion process for guided tissue regeneration. Biomaterials. 1998 November;19(21):1945-55.) The device may optionally be fitted after the tract has been cleaned out by curetting and the scaffold may be pre-seeded with autologous cells to promote tissue healing.

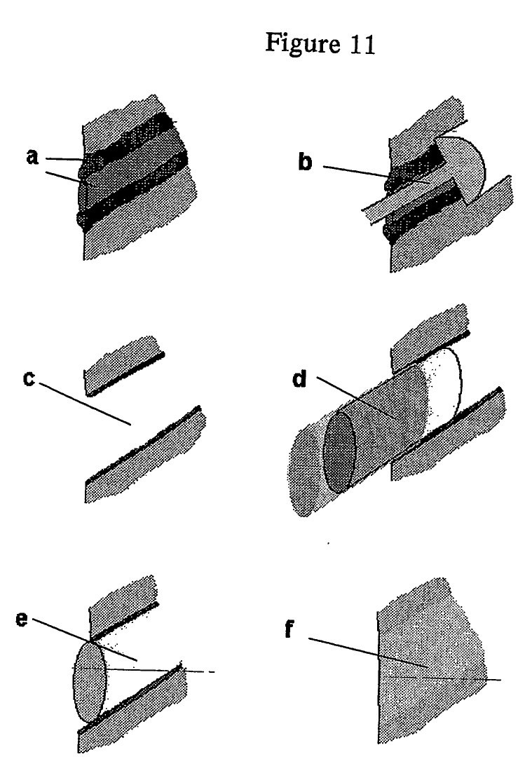

FIG. 11 shows how the device could be applied following cleaning of the fistula tract with a curetting device. The fistula tract lined by granulation tissue (a) is cleaned-out using a curetting device (b) producing a clean tract (c), the device is passed into the tract with the aid of a catheter (d); the latter is withdrawn leaving the device filling the tract (e). Surrounding tissue infiltrates the scaffold, which eventually degrades leaving a fully healed fistula (f).

An alternative method of preparing a tissue construct according to the second aspect of the invention wherein the porous biocompatible polymer is preferably infiltrated with a bioactive material is by using electrophoretic deposition (EPD) (Development and in vitro characterisation of novel bioresorbable and bioactive composite materials based on polylactide foams and Bioglass for tissue engineering applications. Roether J A, Boccaccini A R, Hench L L, Maquet V, Gautier S, Jerjme R. Biomaterials. 2002 September;23(18):3871-8.). This technique, which relies on the presence of charged ceramic particles in a suspension, is known for coating and infiltrating ceramic particles onto a conducting or non-conducting porous and fibrous substrate, as reviewed elsewhere (Use of electrophoretic deposition in the processing of fibre reinforced ceramic and glass matrix composites. A review Boccaccini A R, Kaya C, Chawla K K. Composites A 2001;32:997-1006.).

A natural polymer derived from an extracellular matrix could optionally be used as a biocompatible polymer in the tissue constructs according to the invention. The extracellular matrix (ECM) exists in all tissues and is a complex mixture of a structural and/or functional protein, a glycoprotein, and a proteoglycan arranged into a tissue specific three-dimensional structure. A variety of proteins (including collagen, fibronectin, fibrinogen, laminin, entactin, and/or vitronectin) and glycosaminoglycans (e.g. heparin, chondroitin sulphate, heparan sulphate, hyaluronic acid) found in the ECM support tissue reconstruction. An advantage of using a natural polymer derived from an ECM is that ECM acts as a reservoir for growth factors and cytokines that can modulate cell behaviour. An ECM can be harvested for use as a therapeutic scaffold from the dermis of the skin, submucosa of the small intestine and/or urinary bladder, pericardium, basement membrane and stroma of the decellularized liver, and the decellularized Achilles tendon. An example of an ECM scaffold currently available for use in humans include a porcine heart valve, decellularized and cross-linked human dermis (Alloderm™), acellular porcine collagen (Permacol™) and chemically cross-linked purified bovine type 1 collagen (Contigen™). An example of an ECM scaffold currently available for use in vetinary practice include porcine urinary bladder extracellular matrix (ACell Vet™).

Skin Grafting Preparations

The tissue scaffolds according to the first, second and/or third aspects of the invention are optionally in the form of an intermediate dressing suitable for preparing a wound bed for skin grafting. Particularly such an intermediate dressing includes a synthetic and/or natural polymer coated and/or impregnated with a bioactive material.

The constructs according to the invention preferably include one or more biological cells, preferably an autologous biological cell. A suitable biological cell type for inclusion in a construct according to the invention is a fibroblast or an endothelial cell, for example. The density of the cells on the construct will depend upon the application but a suitable amount might be up to 2 million cells/cm³, depending on the porosity of the polymer.

Drug discovery and development consist of a series of processes starting with the demonstration of pharmacological effects using in vitro cell culture models. Whilst cell culture models can be efficiently used to assess cellular metabolism, cytotoxicity and genotoxicity, the cultures are usually a single phenotype and do not represent the various cell populations found in organs. Therefore in vitro biocompatibility testing may also include the use of ex vivo isolated and perfused organ models. An alternative to these models could be constructed by seeding a tissue construct according to the invention with cells derived from the organ of interest, producing a hybrid organ that could be tested. Constructs coated with bioactive material would ensure optimal vascularization of the tissue construct before being seeded with cells from the organ of interest, e.g. lung cells, intestinal cells, heart cells etc.

The tissue constructs according to the invention are also generally suitable for use in a tissue microarray for use in a high throughput screening test. Presently the tissue used in a high through put screen is generally donated tissue. Use of tissue constructs according to the invention will allow tissue to be synthesised in vitro for use in a tissue microarray for screening for a wider range of targets.

A patient to be treated by the invention is preferably a human or animal (preferably a mammal) patient.

Microencapsulation

According to the invention there is also provided a therapeutic agent delivery system which comprises a biocompatible semi-permeable membrane which encapsulates one or more biological cells and a bioactive material which is as defined above. The therapeutic agent is preferably an angiogenic growth factor, especially VEGF. The membrane preferably permits exchange of nutrients, oxygen and a biologically active product. The membrane is preferably provided by a biocompatible polymer, especially a biocompatible natural polymer, more preferably a biocompatible natural polysaccharide. More preferably the membrane is an alginate, alginate-agarose or alginate-poly-L-lysine.

Cell microencapsulation is a strategy for the controlled, localized and long-term delivery of therapeutic peptides to the host in vivo. This involves the encapulation of cells within semi-permeable devices, such as alginate or alginate-agarose or alginate-poly-L-lysine, which allows the free exchange of nutrients, oxygen and biologically active products between the entrapped cells and the host. Microencapsulated cells have been used for the release of cytokines, hormones, and other agents for gene therapy (Lanza, R. P. et al Encapsulated cell technology. Nat. Biotechnol. 1996; 14:1107-1111; Ismail, N. et al Growth hormone gene therapy using Encapsulated myoblast. In Cell encapsulation technology and therapeutics. (eds Kuhetreiber, W. M., Lanza, R. P. & Chick, W. L.) 343-350 (Birkhauser, Boston, Mass.; 1999); Machluf, M. et al Controlled release of therapeutic agents: slow delivery and cell encapsulation. World J. Urol. 2000; 18:80-83; Savelkoul, H. F. J. et al. Modulation of systemic cytokine levels by implantation of alginate-encapsulated cells. J. Immunol. Methods 1994; 170:185-196; Machluf, M. et al Characterization of microencapsulated liposome systems for the controlled delivery of liposome associated macromolecules. J. Contr. Release 1996; 43:35-45; Prakash, S. et al Microencapsulated genetically engineered live E. coli DH5 cells administered orally to maintain normal plasma urea level in uremic rats. Nat. Med. 1996; 2:883-887), as well as a potential treatment strategy for cancer (Read, T. A. et al Local endostatin treatment of gliomas administered by microencapsulated producer cells. Nat. Biotechnol. 2001; 19:29-34; Orive, G. et al Microencapsulation of an anti VE-cadherin antibody secreting 1B5 hybridoma cells. Biotechnol. Bioeng. 2001; 76:285-294; Joki, T. et al Continuous release of endostatin from microencapsulated engineered cells for tumor therapy. Nat. Biotechnol. 2001; 19:35-39).

Microencapsulation of fibroblasts, or other cell types, with an optimum amount of bioactive material as defined above, in a semi-permeable membrane, such as alginate or alginate-agarose or alginate-poly-L-lysine, allows the free exchange of biologically active products secreted between the encapsulated cells and the host. This provides a prolonged sustained delivery of VEGF and other angiogenic growth factors by cells.

The invention is illustrated by way of example with reference to the Figures of the accompanying drawings of which:

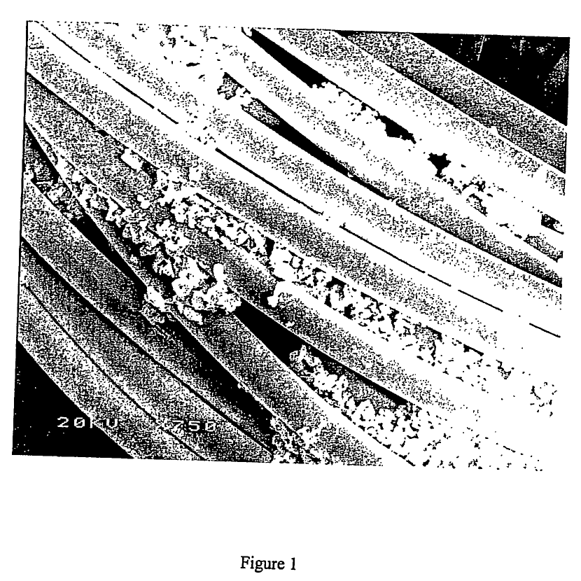

FIG. 1 is a SEM micrograph of a polyglycolic acid (PGA) mesh coated with 45S5 Bioglass®. Glass particles can be seen on and between the woven mesh fibres (original magnification ×750);

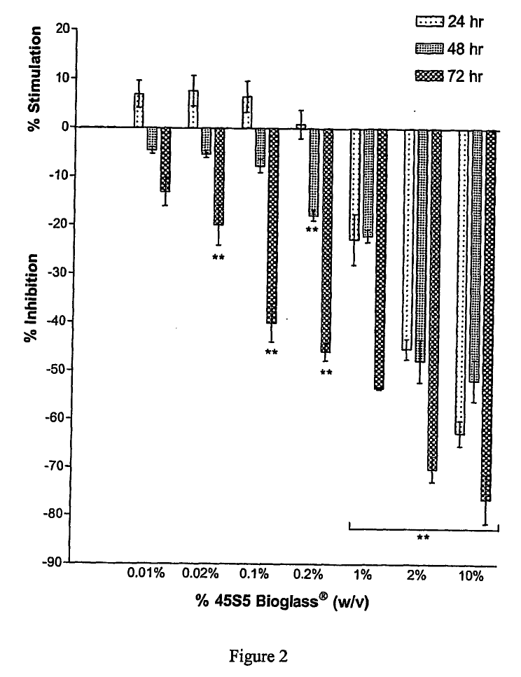

FIG. 2 is a graph showing the effect of different concentrations of 45S5 Bioglass® on fibroblast proliferation after 24, 48 and 72 hours in culture. The percentage change in cell proliferation in response to 45S5 Bioglass® was calculated relative to cells growing in the absence of 45S5 Bioglass® for each time point. Data are means of triplicate experiments. The vertical bars are the standard deviations (** p<0.01).

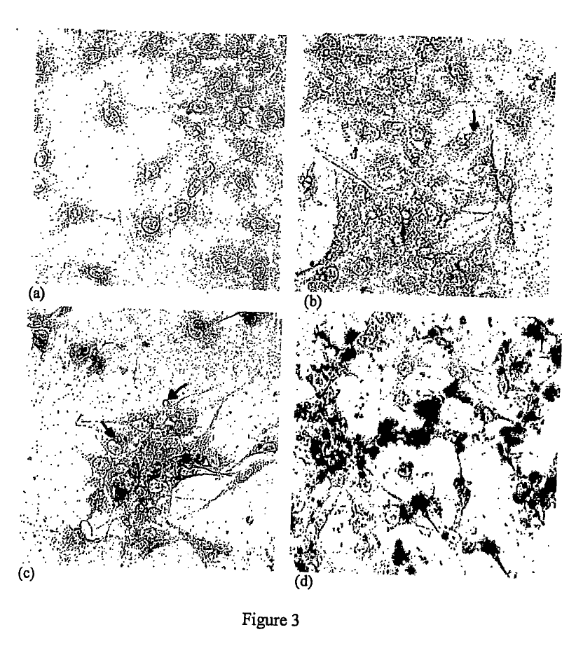

FIG. 3 is a representation of four micrographs (Wright's Giemsa; original magnification ×400) of light microscopy of 208F fibroblasts grown on surfaces coated with different concentrations of 45S5 Bioglass® for 24 hours. Micrograph (a) is of 208F cells grown on control surfaces in the absence of 45S5 Bioglass® and shows uniform spreading of the cytoplasm with no vacuoles. Micrographs (b) and (c) show the morphology of 208F cells grown on surfaces coated with 0.01% and 0.02% 45S5 Bioglass®, respectively; this morphology demonstrates more elongated lamellipodia and the presence of cytoplasmic vacuoles. Micrograph (d) shows that cells grown on 0.1% 45S5 Bioglass® had increased spindle-like projections.

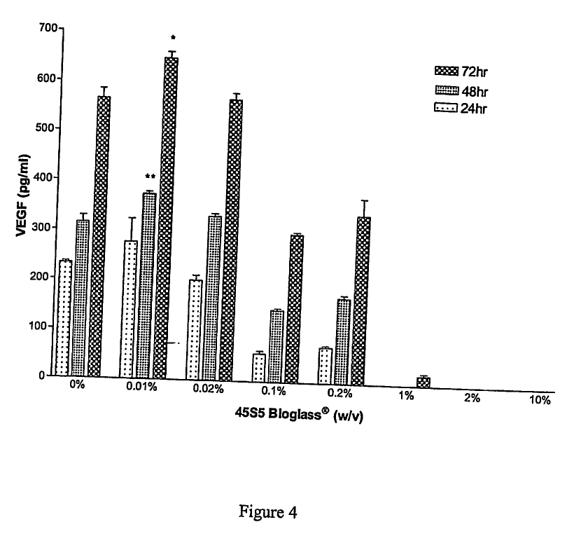

FIG. 4 is a graph showing the degree of VEGF secretion by 208F fibroblasts grown on 45S5 Bioglass®-coated cell culture plates as assessed in conditioned culture medium collected after 24, 48 and 72 hours. Data are means of triplicate experiments. The vertical bars are the standard deviations (*p<0.05; **p<0.01).

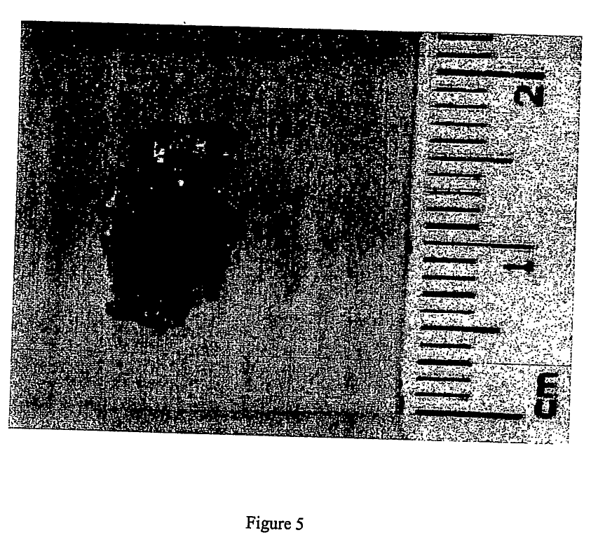

FIG. 5 is a photograph of excised 45S5 Bioglass® composite mesh at 42 days viewed enface. The implanted meshes were completely encapsulated by new tissue at each of the time points examined.

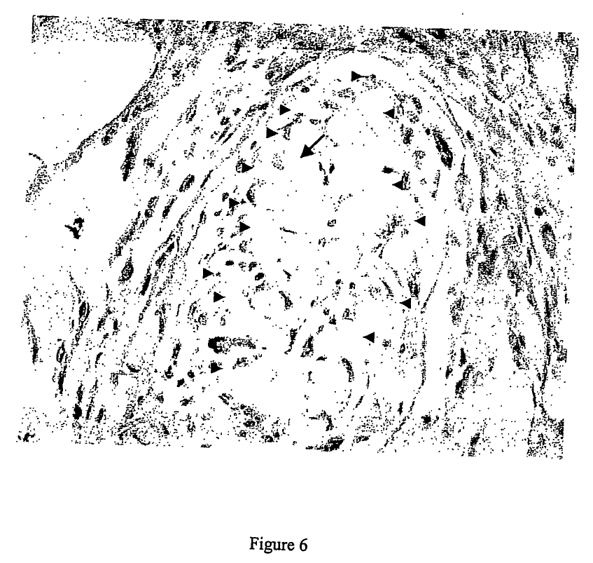

FIG. 6 is a photograph (Haematoxylin and van Gieson staining; original magnification ×400) showing that PGA/45S5 Bioglass®-composite meshes were fully cellularized at 14 days. Collagen (stained portions) is deposited between the woven mesh fibre (arrowheads) and blood vessels (arrows).

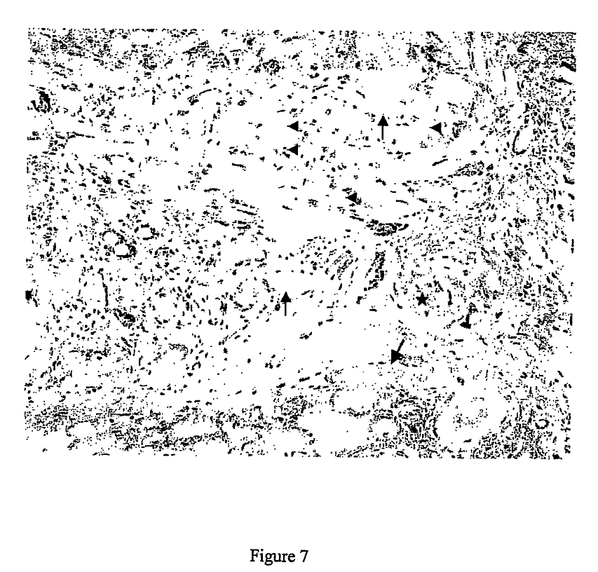

FIG. 7 is a photograph (Haematoxylin and van Gieson staining; original magnification ×200) of blood vessels (arrows) with erythrocytes present in the lumen which are scatted throughout the 45S5 Bioglass®-composite mesh (arrowheads) and the surrounding tissue (42 days).

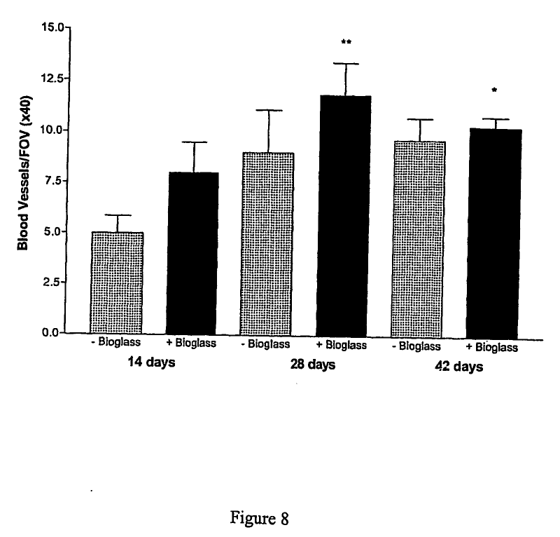

FIG. 8 is a graph of the number of blood vessels counted in six random fields of view (FOV) at ×400 magnification. Significantly more vessels were present in PGA/45S5 Bioglass®-composite meshes at 28 days (**p<0.01) and 42 days (*p<0.05) compared with uncoated meshes at 14 days;

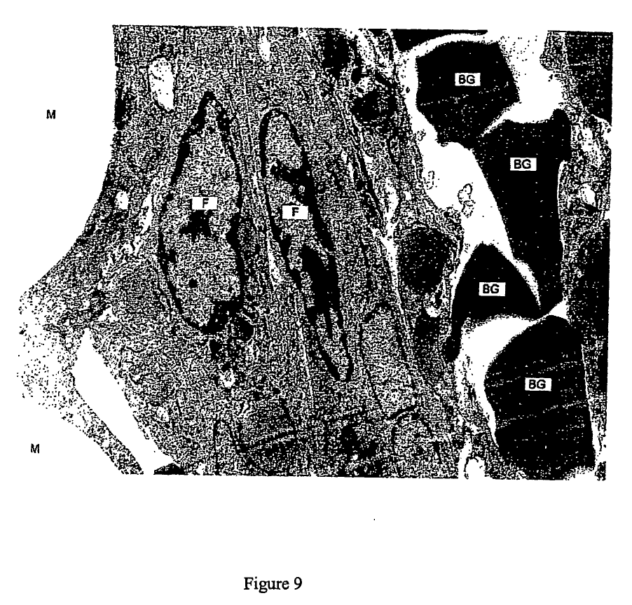

FIG. 9 is an electron micrograph (original magnification ×13,800) of fibroblasts (F) in the neotissue closely adhering to the PGA mesh fibres (M) after 14 days. Spaces exist between cells in areas containing aggregates of 45S5 Bioglass® (BG);

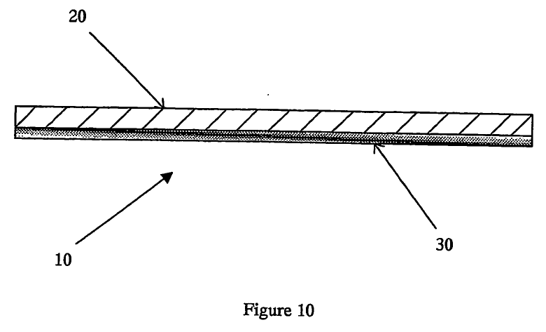

FIG. 10 is a schematic cross-sectional view of a wound dressing according to the invention;

FIG. 11 illustrates in schematic form how a tissue construct according to the invention which is in the form of a porous biocompatible membrane containing bioactive material is applied to a fistula tract following cleaning with a curetting device;

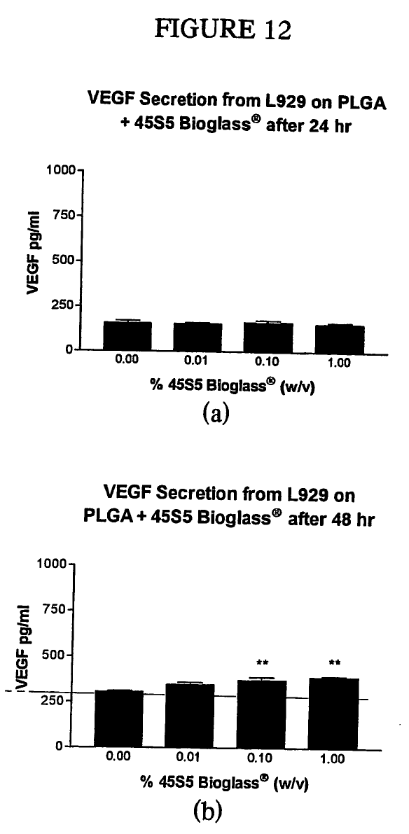

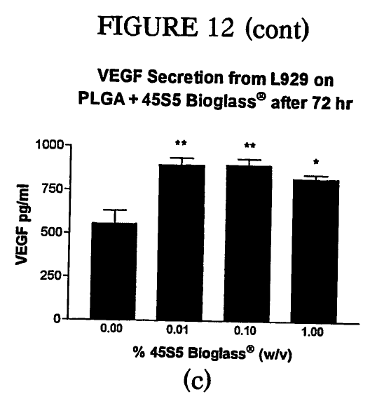

FIG. 12 is a graph showing the amount of VEGF secretion from L929 fibroblasts on PLGA and the given amounts of 45S5 Bioglass after 24 hours (a), 48 hours (b) and 72 hours (c);

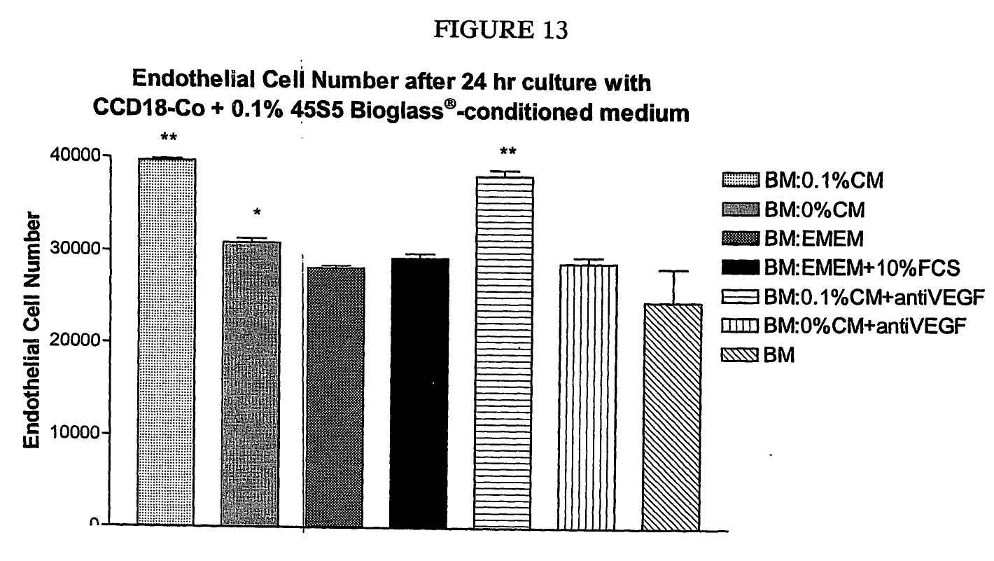

FIG. 13 is a graph showing the endothelial cell number after human dermal microvascular endothelial cells had been stimulated with conditioned culture medium collected from human colonic fibroblasts (CCD-18co) which had been cultured on surfaces coated with 0% or 0.1% (w/v) 45S5 Bioglass and then cultured for 24 hours; and

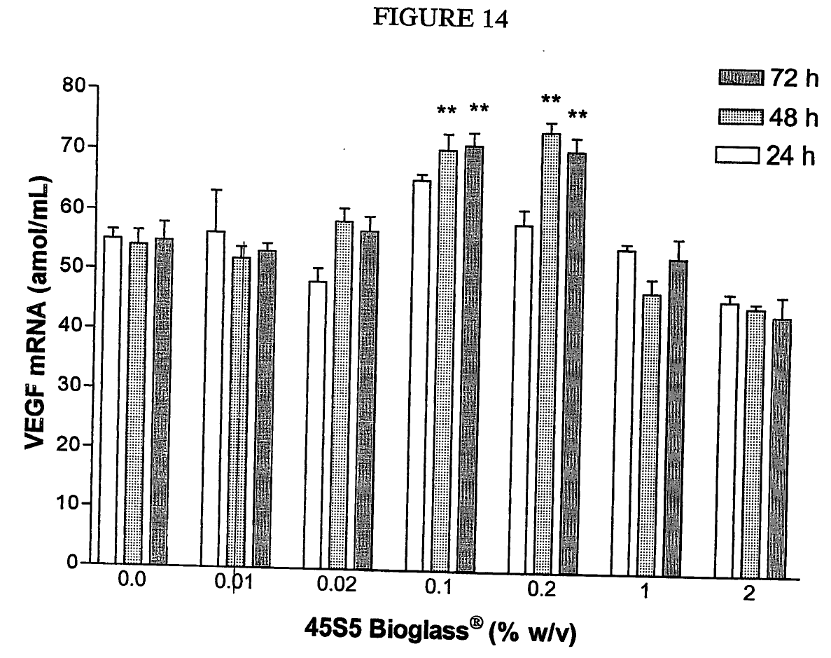

FIG. 14 is a graph showing the amount of VEGF mRNA produced by CCD-18co cells incubated on culture plates coated with different concentrations of 45S5 Bioglass;

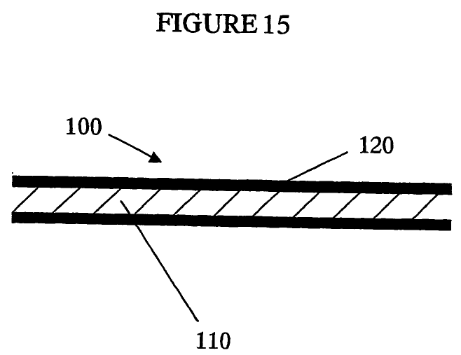

FIG. 15 is a schematic representation of a suture according to the invention in the form of a surgical staple;

FIG. 16 shows digital micrographs of human endothelial cells grown in (a) Optimised medium alone; (b) optimised medium+2 ng/ml VEGF; (c) optimised medium+20 μM suramin; (d) optimised medium:conditioned medium (0 g/cm²); (e) optimised medium:conditioned medium (0.3125 mg/cm²). After 11 days of culture the endothelial cells proliferated and migrated to form an anastomosing network of newly formed endothelial tubules. Images shown are representative of the mean values obtained from the image analysis. Original magnification ×4; and

FIG. 17 shows graphs of the data obtained from image analysis of FIG. 16 wherein (a) shows number of junctions formed; (b) number of tubules formed; (c) total tubule length; (d) mean tubule length. Conditioned medium from fibroblasts grown on 45S5 Bioglass® produced a significant increase to the number of endothelial tubules (p<0.05), total tubule length (p<0.01) and number of tubule junctions (p<0.05). (*p<0.05, **p<0.01).

FIG. 10 shows a wound dressing 10 having a dressing layer 20 and a bioactive material layer 30. In use, the wound dressing could be applied to a wound by using a bandage.

FIG. 15 shows a suture 100 having a thread 110 coated with a layer of bioactive material 120.

The invention is illustrated by the following Examples, which are not intended to limit the scope of the application.

### EXAMPLE 1

In the following Example, 45S5 Bioglass® was assessed for its application in soft tissue engineering. The effect of 45S5 Bioglass® on cell proliferation, spreading and growth factor secretion was investigated in vitro using fibroblast cell lines.

Polyglycolic acid mesh in the form of a sterile knitted sheet (Dexon mesh®) was purchased from Sherwood, Davis & Geck, Gosport, UK. The bioactive material used was a melt-derived bioactive glass (BG) powder (45S5 Bioglass®, US Biomaterials Co., Alachua, Fla., USA). The powder had a mean particle size <5 μm. The composition of the bioactive glass used was (in wt. %): 45% SiO₂, 24.5% Na₂O, 24.5% CaO and 6% P₂O₅, which is the original composition developed by Hench and co-workers in 1971 (Hench L L, Splinter R J, Allen W C, Greenlee T K. Bonding mechanisms at the interface of ceramic prosthetic materials. J. Biomed. Mater. Res. 1971; 2:117-141).

Cells and Cell Culture Conditions

Fibroblasts (208F) derived from a sub-clone of a Fischer rat fibroblast 3t3-like line RAT-1 were maintained in Eagle's Minimum Essential Medium supplemented with 2 mM glutamine, 1% non-essential amino acids, 1% vitamins and 10% FBS. Cells were subcultured weekly after short treatment with 0.05% trypsin/0.02% EDTA in Hank's balanced salt solution and cultured in 5% CO₂ at 37° C.

Coating of Culture Surfaces with 45S5 Bioglass® for In Vitro Assessment of Cell Proliferation, Morphology and Growth Factor Secretion

Because there has been limited previous research on the use of 45S5 Bioglass® in soft tissue engineering, a wide range of concentrations of 45S5 Bioglass® were studied for their effect on fibroblasts in vitro. Slurries of 45S5 Bioglass® (10, 2, 1, 0.2, 0.1, 0.02, 0.01% (wt/vol) were prepared in distilled and deionised water. For proliferation assays, one hundred microlitres of each solution was added to individual wells of a 96-well polystyrene cell culture plate (Costar Corp., NY, USA). For measurement of growth factor secretion cells were seeded onto a 24-well plate polystyrene cell culture plate (Costar Corp.). To compensate for the differences in growth surface area between plates, the amount of 45S5 Bioglass® added to each well of the 24-well plate was increased by a ratio of 5.94. To assess cell morphology, glass microscope slides, sterilized by autoclaving, were covered with a flexiPERM chamber (Sigma). To compensate for the differences in growth surface area between 96-well plates and the flexiPERM chamber, the amount of 45S5 Bioglass® added to each well of the chamber was reduced by a ratio of 1.14. The plates and slides were allowed to air dry in a laminar air-flow hood leaving a thin coating of Bioglass® at the base of each well (Table 1).

TABLE 1

| | | | | | | | |
|---|---|---|---|---|---|---|---|
| 45S5 Bioglass ® % wt/vol | 10% | 2% | 1% | 0.2% | 0.1% | 0.02% | 0.01% |
| mg/cm² | 31.25 | 6.25 | 3.125 | 0.625 | 0.3125 | 0.0625 | 0.03125 |
| Volume added to 96-well plate | 100 μl | 100 μl | 100 μl | 100 μl | 100 μl | 100 μl | 100 μl |
| Volume added to 24-well plate | 594 μl | 594 μl | 594 μl | 594 μl | 594 μl | 594 μl | 594 μl |
| Volume added to flexiPERM | 87.5 μl | 87.5 μl | 87.5 μl | 87.5 μl | 87.5 μl | 87.5 μl | 87.5 μl |

Cell Proliferation

Suspensions of 208F cells (3×10⁴ cells/ml) were seeded onto the 96-well cell culture plates coated with different concentrations of 45S5 Bioglass® in triplicate. The plates were incubated for 24, 48 or 72 hours in 5% CO₂ at 37° C. After incubation changes in cell proliferation were determined using a CytoTOX 96 cytotoxicty assay (Promega, UK), a non-radioactive colorimetric assay that quantitatively measures lactate dehydrogenase, a stable cytosolic enzyme. The assay was performed according to manufacturer's instructions. The percentage change in cell proliferation was calculated from the absorbance values for cells cultured in wells coated with 45S5 Bioglass® relative to cells cultured in control wells without a coating of 45S5 Bioglass®.

Cell Spreading

Suspensions (1×10⁴ cells/ml) of 208F cells were added to the glass microscope slides coated with 45S5 Bioglass® covered with a flexiPERM chamber. The slides were incubated for 24 hours in 5% CO₂ at 37° C. After incubation, the flexiPERM chamber was removed and the slides were rinsed in PBS, fixed in chilled methanol and stained with Wright's Giemsa solution. The slides were allowed to air-dry before being examined using a light microscope.

Measurement of Growth Factor Secretion

Suspensions of 208F cells (5×10⁴ cells/ml) were seeded onto 24-well culture plates coated with different concentrations of 45S5 Bioglass® in triplicate. The plates were incubated for 24, 48 or 72 hours in 5% CO₂ at 37° C. After incubation the conditioned culture medium was collected and immediately stored at −70° C. The amount of VEGF present in the conditioned medium was determined by using a quantitative sandwich enzyme immunoassay (Quantikine®M mouse VEGF; R&D Systems, UK). This assay is primarily designed for the quantitative determination of mouse VEGF but also cross-reacts with rat VEGF (information obtained from R&D Systems, UK). Negative controls included in the experiment were medium collected from cells seeded on surfaces that were not coated with 45S5 Bioglass®, and culture medium+10% FBS without cells. A positive control was included in the assay, provided by the manufacturer and known to contain between 96-160 pg/ml VEGF.

Results

Cell Proliferation

Cell proliferation in response to different surface coating concentrations of 45S5 Bioglass® was assessed at 24, 48 and 72 hours after seeding with 208F fibroblasts using the CytoTOX 96 cell proliferation assay. At 24 hours, 45S5 Bioglass®-coated surfaces stimulated cell proliferation at concentrations of 0.01% (mean increase 6.85±4.76%), 0.02% (mean increase 7.58±5.41%), 0.1% (mean increase 6.45±5.52%) and 0.2% (mean increase 0.88±5.06%) compared with untreated surfaces. Surfaces coated with concentrations higher than 0.2% significantly inhibited cell proliferation compared with untreated control cells (p<0.01). At 48 and 72 hours, all concentrations of 45S5 Bioglass® inhibited fibroblast proliferation compared with untreated control cells, significantly so for concentrations above 0.2% at 48 hours (p<0.01) and concentrations above 0.02% at 72 hours (p<0.01) (FIG. 2).

This effect of inhibited fibroblast proliferation did not appear to result from a build up of inhibitory factors in the conditioned medium because a similar pattern was observed when the culture medium was replaced with fresh medium at the end of each 24-hour interval (data not shown). The reduced fibroblast proliferation in response to 45S5 Bioglass® could result from a shift towards a more differentiated cell phenotype, which could also account for the increase in growth factor secretion.

Cell Morphology

Cell morphology and spreading were studied after the cells had been attached to surfaces coated with 45S5 Bioglass® for 24 hours (FIG. 3). Cells grown on uncoated surfaces had uniformly spread cytoplasm containing no vacuoles (FIG. 3a). Cell morphology at 0.01%-0.02% 45S5 Bioglass® was similar to control cells except that the cytoplasm of some cells contained small vacuoles. Also some cells were stretched out further producing spindle-like lamellipodia (FIG. 3b-3c). At concentrations of 0.1%-0.2% 45S5 Bioglass® the spindle-like projections were more elongated and the cytoplasm appeared to be less spread-out. The morphology of cells at 1%-10% 45S5 Bioglass® was not visible because the transmission of light was blocked by the surface covering of 45S5 Bioglass®.

Growth Factor Secretion

VEGF production by 208F fibroblasts was assessed in conditioned culture medium collected after 24, 48 and 72 hours culture in 45S5 Bioglass®-coated 24-well cell culture plates. Values are shown after subtracting the amount of VEGF measured in cell culture medium plus 10% FCS. Fibroblasts grown on 0.01% 45S5 Bioglass® had increased secretion of VEGF into the culture medium compared with control fibroblasts grown on uncoated surfaces at 24 hours (mean 276.7±82.02 pg/ml versus 232.9±5.28 pg/ml), 48 hours (mean 373.8±9.14 pg/ml versus 315.5±24.25 pg/ml; p<0.01) and 72 hours (mean 647.5±22.24 pg/ml versus 566.3±34.10 pg/ml; p<0.05). The amount of VEGF secreted into the medium by fibroblasts grown on 0.02% 45S5 Bioglass® was also increased at 48 hours (mean 332.0±9.61 pg/ml versus 315.5 pg/ml) and 72 hours (mean 568.1±21.9 pg/ml versus 566.3 pg/ml). Concentrations of 45S5 Bioglass® greater than 0.1% inhibited secretion of VEGF from fibroblasts compared with control fibroblasts grown on uncoated surfaces (FIG. 4). Cell culture medium plus 10% FCS was found to contain on average 31.8±0.95 pg/ml of VEGF. The positive control contained on average 129.19±11.69 pg/ml of VEGF.

### EXAMPLE 2

In the following Example, an in vivo animal model was used to assess the response to polyglycolic acid (PGA)/45S5 Bioglass® composite meshes that were allowed to cellularize for 14, 28 or 42 days before being removed and assessed by histology using light and electron microscopy.

Fabrication of PGA/45S5 Bioglass®-Composite Mesh

A stable slurry of 40% (wt/vol) 45S5 Bioglass® particles in distilled and deionised water was prepared. One-centimetre squares of PGA mesh were lowered into the slurry using tweezers and left in immersion for 5 min with continuous stirring. Following immersion, the samples were carefully withdrawn to avoid damage at a withdrawal velocity of ˜5 cms⁻¹. The samples were subsequently dried on glass plates at room temperature in a humid-controlled atmosphere. To restore the porosity of the mesh that had been obscured by 45S5 Bioglass® particles, a pipette tip was rolled across the surface of the coated mesh whilst being simultaneously flexed. This resulted in a thin coating of 45S5 Bioglass® on the PGA mesh fibres (FIG. 1). Samples were routinely processed for scanning electron microscopy. The amount of 45S5 Bioglass deposited on the mesh after soaking was difficult to quantify since the depostion was not uniform, as FIG. 1 shows. According to previous results on similar materials, it is assumed that the surface of the meshes was covered in about 20% with Bioglass particles. At least 5 meshes were examined by SEM previous to implantation and all exhibited a similar, rather uniform coating of Bioglass particles, which indicated the good reproducibility of the coating method. The meshes were sterilized by placing them in a tissue culture laminar flow hood equipped with a UV lamp. The samples were sterilized under UV light for 30 minutes on either side. This method of sterilization has been used in previous studies on similar composite scaffolds.

Implantation of 45S5 Bioglass®/PGA Composite Mesh

Implantation studies were performed on inbred adult male Lewis rats weighing 250-350 g in compliance with the Animals (Scientific Procedures) Act 1986. All the animals were fed on commercial standard pelleted rat diet. Rats were anaesthetised with Hypnorm 0.4 ml/kg (fentanyl citrate and fluanisone) and diazepam 5 mg/kg. Six implants (three PGA mesh, three PGA mesh/45S5 Bioglass® composite meshes) that had been sterilized by ultraviolet light were placed in a subcutaneous pocket on the ventral aspect of each rat and closed with 3/0 Mersilk® sutures (Ethicon®). Rats were kept under standard laboratory conditions until sacrifice at 14, 28 and 42 days, when the meshes were removed.

Histological Examination of Implanted 45S5 Bioglass®/PGA Composite Mesh

For light microscopy, the tissues containing the encapsulated meshes were fixed in 10% buffered formalin. During embedding into paraffin-wax the meshes were orientated so that a cross-section of the mesh would be cut during sectioning for histological examination. Five-micrometer tissue sections were cut and stained with haematoxylin and van Gieson stain. The number of blood vessels present in each mesh was counted under a light microscope (magnification 400×) in six different randomly selected fields within the mesh for each sample. Blood vessels were identified by the inclusion of erythrocytes within the blood vessel lumen. The counting was conducted in a blinded manner with regards to the identification of PGA mesh/45S5 Bioglass® composites or control samples. For transmission electron microscopy (TEM) the tissues were fixed in 2.5% glutaraldehyde-phosphate buffer, post-fixed in osmium tetroxide and dehydrated in acetone before being embedded in araldite resin. Ultrathin sections (70-90 nm) were positively stained with uranyl acetate and lead citrate before being examined using a Jeol JEM 1200 EX transmission electron microscope.

Statistical Analysis

All results are expressed as means±SD for experiments repeated in triplicate unless otherwise stated. The level of statistical significance was measured by one-way analysis of variance with post hoc Dunnet's test is specified in the text. Statistical significance was accepted at the p<0.05 level.

Cellularization of PGA/45S5 Bioglass® Composite Meshes

Both PGA mesh and PGA mesh/45S5 Bioglass® composites were completely encapsulated by new tissue at all of the time points studied (FIG. 5). Under light microscopy, the woven mesh was completely cellularized mainly by fibroblasts but some macrophage-like cells were also scattered throughout the tissue. Haematoxylin and van Gieson staining revealed deposition of collagen in and around the mesh (FIG. 6). The meshes were well vascularized with neovascularization increasing throughout the duration of the study (FIG. 7).

The number of blood vessels counted per field of view at ×400 magnification was significantly increased at 28 (p<0.01) and 42 (p<0.05) days in the PGA/45S5 Bioglass® composite mesh compared with the number of blood vessels in the uncoated mesh at 14 days. No significant increase was seen in uncoated meshes at the same time-points (FIG. 8).

TEM of the implanted PGA/45S5 Bioglass® mesh composites demonstrated fibroblasts closely adhering to the PGA mesh. In areas that contained aggregates of 45S5 Bioglass® particles small spaces were visible between the glass particles and the surrounding cells (FIG. 9).

### EXAMPLE 3

In the following Example, the amount of VEGF secreted by L929 fibroblasts grown on PLGA/45S5 Bioglass composite films was assessed.

To prepare a polymer composite film, 0.5 g PLGA (Medisorb 7525 DL Low IV Alkermes, USA) was dissolved in 10 ml chloroform (5% w/v). The PLGA solution was then filtered through a 0.22 μm filter. 0.02 g 45S5 Bioglass® was added to 2 ml PLGA solution (1% w/v) and the solution was mixed by vortexing and sonication for 15 minutes. 1% (w/v) solution was diluted 10-fold in PLGA solution to produce a 0.1% (w/v) solution and the 0.1% (w/v) solution was diluted 10-fold in PLGA solution to produce a 0.01% (w/v) solution.

40 μl of PLGA/45S5 Bioglass® solution cast onto individual 13 mm diameter glass coverslips that had been previously cleaned by immersion in 70:30 ethanol:ether. Chloroform was allowed to evaporate off in a laminar air-flow hood leaving a film of PLGA/45S5 Bioglass® attached to the coverslip.

It should be noted that although the amount of Bioglass® quoted for the preparation of a polymer composite film is % wt/vol, the quantity of 45S5 Bioglass® present in the films is different from the amount in the tissue culture plates coated with 45S5 Bioglass® alone. This is because after the solvents used have evaporated, the surface of the polymer composite, based on an initial 5% w/v polymer/solvent mix, will include both PLGA and Bioglass®, whereas the surface of the tissue culture plate will have Bioglass® alone.

Assessment of VEGF Secretion from L929 Fibroblasts Grown on PLGA/45S5 Bioglass® Composites Films:

A suspension of mouse fibroblasts (L929) was seeded at 5×10⁴ cells/well for each concentration of Bioglass®/polymer in triplicate. The plates were incubated at 37° C. in a humidified atmosphere of 5% CO₂ for 24, 48, 72 hr. The amount of VEGF secreted into the conditioned culture medium was measured using a Quantikine M mouse VEGF ELISA (R&D Systems, cat no. MMV00). The results are shown in FIG. 12.

A significant (p<0.01) increase in VEGF secretion occurs after 48 hr at 0.1 and 1% (w/v) 45S5 Bioglass®. After 72 hr a significant increase in VEGF secretion occurs at 0.01, 0.1 (both p<0.01) and 1% (p<0.05) (w/v) 45S5 Bioglass®. A significant increase in secretion of VEGF is seen at higher concentrations of 45S5 Bioglass® when used as a polymer composite compared with tissue culture plates coated with 45S5 Bioglass® alone. This is probably due to relatively smaller quantities of the Bioglass® being exposed to cells when it is mixed with polymer, making the range of optimal concentrations for polymer composites include larger amounts of Bioglass® (up to 1% w/v).

### EXAMPLE 4

In the following Example, to demonstrate that the products secreted from fibroblasts cultured on 45S5 Bioglass® produce an angiogenic response rather than an angiostatic response, conditioned medium from the fibroblasts was used to stimulate the human dermal microvascular endothelial cells.

Human dermal microvascular endothelial cells from a 30 year-old female (ZHC-2111; TCS Cell Works) grown in microvascular endothelial cell basal medium (ZHM-2946; TCS Cell Works) containing microvascular endothelial cell growth supplement (ZHS-8947; TCS Cell Works) and antibiotic supplement (ZHR-9939; TCS Cell Works) were seeded into a 96 well plate (previously coated with attachment factor [ZHS-8949; TCS Cell Works]) at 1×10⁴ cells per well.

After 24 hr the medium was removed from each well. The endothelial cells were stimulated with conditioned culture medium (CM) collected from human colonic fibroblasts (CCD-18co) that had been cultured on surfaces coated with 0% or 0.1% (w/v) (0 g/cm² or 0.3125 mg/cm², respectively) 45S5 Bioglass® for 72 hours. The conditioned medium was diluted 1:1 with endothelial basal medium (BM) and 100 μl of the solution was added to wells containing endothelial cells in triplicate. Controls included cells stimulated with BM alone, BM plus CCD-18co culture medium (EMEM) (+/−10% fetal calf serum). 2 μg/ml anti-human VEGF monoclonal antibody was added to additional wells containing 0.1% or 0% CM:BM in an attempt to block any stimulatory effect produced by VEGF. The endothelial cells were cultured for 24 hr and the changes to cell number were determined using the Cell Titer 96 Assay (Promega). The absorbance readings were adjusted to cell numbers using a standard curve created with the Cell Titer 96 Assay and known numbers of endothelial cells.

The results are shown in FIG. 13. From these data, it can be seen that if the endothelial cell proliferation response of stimulated cells is compared with endothelial cells cultured with basal medium alone, the response that the 45S5 Bioglass®-k/fibroblast-conditioned medium produced a greater significant increase in endothelial cell numbers (p<0.01) compared with endothelial cells cultured with conditioned medium produced by fibroblasts alone (p<0.05). The presence of anti-VEGF antibody did not reduce the significant response, indicating that other angiogenic growth factors, which are not effectively blocked by this antibody, are present to stimulate the proliferation of endothelial cells.

### EXAMPLE 5

To evaluate the angiogenic growth factors secreted from fibroblasts in response to 45S5 Bioglass®, a commercial angiogenesis assay (Angiogenesis Assay Kit; TCS CellWorks Ltd) was used (Donovan D et al. Comparison of three in vitro human ‘angiogenesis’ assays with capillaries formed in vivo. Angiogenesis. 2001;4:113-121; Drinkwater S L et al Effect of venous ulcer exudates on angiogenesis in vitro. Br J Surg. 2002;89:709-713; Sengupta S et al. Thymidine phosphorylase induces angiogenesis in vivo and in vitro: an evaluation of possible mechanisms. Br J Pharmacol. 2003;139:219-231). The assay was performed according to the manufacturer's instructions.

In brief, on the day of delivery from the supplier, the medium in the culture plate was replaced with optimised medium (TCS CellWorks Ltd; supplied with the kit) alone (OM); OM:conditioned medium (medium collected from human colonic fibroblasts (CCD-18co) cultured on 0 g/cm² or 0.3125 mg/cm² 45S5 Bioglass® for 72 hours) at 1:1 v/v; OM containing 2 ng/ml VEGF (positive control); or OM containing 20 μM suramin (negative control). Each treatment condition was repeated in quadruplicate. The culture medium was removed on days 4, 7 and 9 of the experiment and replaced with fresh pre-equilibrated (37° C. and 5% CO₂) optimised medium plus each treatment. After 11 days, the endothelial cells were stained for CD31 (TCS CellWorks Ltd) using indirect immunocytochemistry as described in the manufacturer's instructions and allowed to air-dry. Each well was divided into quadrants on the underside of the plate. Photomicrographs of each quadrant were recorded with a Nikon Eclipse TE2000-S inverted photomicroscope fitted with a digital camera using a 4× objective lens. Image analysis software (AngioSys, Foster Findlay Associates, Newcastle Upon Tyne, UK) was used for automated scoring of the assay, providing quantitative measurements of endothelial tubule development (total vessel number, total tubule length and number of junctions), as previously described (Drinkwater S L et al Effect of venous ulcer exudates on angiogenesis in vitro. Br J Surg. 2002;89:709-713; Sengupta S et al. Thymidine phosphorylase induces angiogenesis in vivo and in vitro: an evaluation of possible mechanisms. Br J Pharmacol. 2003; 139:219-231).

After 11 days of culture the endothelial cells were stained for CD31 expression and images of each quadrant recorded as digital photomicrogaphs (FIG. 16). The presence of VEGF (FIG. 16b) and conditioned medium collected from fibroblasts cultured on surfaces coated with 45S5 Bioglass® (FIG. 16e) produced a visible increase to the number of anastomosed endothelial tubules compared with endothelial cells cultured in optimised medium alone (FIG. 16a), suramin (FIG. 16c) or conditioned medium collected in the absence of 45S5 Bioglass® (FIG. 16d).

Image analysis of the micrographs indicates that 45S5 Bioglass® stimulates fibroblasts to secrete growth factors that produce a significant increase in angiogenesis (FIG. 17). Conditioned medium collected from fibroblasts cultured on 45S5 Bioglass® produced a significant increase to the number of endothelial tubules (97.1%, p<0.05; FIG. 17a), total tubule length (93.7%, p<0.01; FIG. 17b) and the number of tubule junctions (210.2%, p<0.05; FIG. 17c) compared with control endothelial cells cultured with optimised medium alone. Significant increases were also produced with the addition of 2 ng/ml VEGF as a positive control for the assay, but not with the conditioned medium collected in the absence of 45S5 Bioglass®. The presence of 20 μM suramin, a known inhibitor of angiogenesis, reduced the number of endothelial tubules, total tubule length and the number of tubule junctions compared with control endothelial cells.

The addition of conditioned medium produced in the presence of 45S5 Bioglass® to the in vitro model of angiogenesis resulted in a visible increase to tubule branching and the formation of a complex network of interconnected tubules, confirmed by the image analysis indicating a significant increase to the number of endothelial tubules and junctions. The development of these tubules mimics many of the stages known to be essential for angiogenesis including cell migration, cell proliferation, vessel branching and anastomosis (Folkman J. Tumor angiogenesis. Adv Cancer Res. 1985;43:175-203).

### EXAMPLE 6

In the following Example, the amount of VEGF mRNA produced by human colonic fibroblasts in response to 45S5 Bioglass® was determined. This is important because the increased amount of VEGF secreted in response to 45S5 Bioglass® may result from either increased secretion of VEGF from intracellular reserves or by regulation of VEGF production at the transcriptional level or by modulation of mRNA stability (Neufeld, G., Cohen, T., Gengrinovitch, S., Poltorak, Z. Vascular endothelial growth factor (VEGF) and its receptors. FASEB J. 13, 9-22 (1999)).

The amount of VEGF mRNA was determined using Quantikine® mRNA Colorimetric mRNA Quantitation assay (R&D Systems) that detects all known human VEGF mRNA splice variants. CCD-18Co cells (5×10⁴ cells/ml) were seeded onto 24-well culture plates coated with different concentrations of 45S5 Bioglass® in triplicate. The plates were incubated for 24, 48 or 72 hours in 5% CO₂ at 37° C. Cell lysates were prepared using the lysis diluent provided with the kit and immediately frozen until the assay. The assay procedure was performed according to the manufacture's instructions. The optical density of each well was measured using a microplate reader (Dynatech MRX) at 490 nm. A calibrator curve, produced using known quantities of RNA calibrator, was used to determine the concentration of mRNA in each sample. The results are shown in FIG. 14.

The increased amount of VEGF measured in the culture medium from cells growing on surfaces coated with between 0.1%-0.2% (w/v) 45S5 Bioglass® correlates with increased quantities of VEGF mRNA at the same range of concentrations, indicating the increase in VEGF secretion may result from de novo synthesis. This suggests that the increase in VEGF secretion produced by 45S5 Bioglass® results from regulation of VEGF production at the transcriptional level.

1. A bioactive material for use in stimulating vascularisation.

2. A material as defined in claim 1 for use in inducing secretion of an endothelial cell mitogen (especially VEGF).

3. A material as defined in claim 1 which comprises SiO₂ and CaO and optionally P₂O₅, CaF₂, MgO, Al₂O₃, TiO₂, phosphate ions, SrO, K₂O, B₂O₃, fluoride ions, Na₂O and/or Ag₂O, preferably Na₂O and/or P₂O₅.

4. A material as defined in claim 1 which comprises:
CaO, SiO₂, P₂O₅, CaF₂;
CaO, MgO, SiO₂, P₂O₅, CaF₂;
CaO, P₂O₅, SiO₂, Al₂O₃, TiO₂;
CaO, P₂O₅, SiO₂, CaF₂;
CaO, phosphate ions, SiO₂;
CaO, Sro, SiO₂, P₂O₅, CaF₂;
K₂O, MgO, Al₂O₃, B₂O₃, SiO₂, CaO, P₂O₅, fluoride ions;
K₂O, MgO, CaO, Al₂O₃, B₂O₃, SiO₂, P₂O₅, fluoride ions;
MgO, CaO, SiO₂;
Na₂O, CaO, MgO, Al₂O₃, SiO₂, P₂O₅, CaF₂;
Na₂O, CaO, P₂O₅, SiO₂;
Na₂O, K₂O, MgO, CaO, B₂O₃, P₂O₅, SiO₂;
SiO₂, Na₂O, CaO, P₂O₅, K₂O, Al₂O₃, MgO;
SiO₂, Al₂O₃, P₂O₅, Na₂O, K₂O, CaO, fluoride ions;
SiO₂, CaO;
SiO₂, CaO, Na₂O, P₂O₅;
SiO₂, CaO, P₂O₅;
SiO₂, CaO, P₂O₅, Ag₂O;
SiO₂, MgO, Al₂O₃, K₂O, CaO, P₂O₅, fluoride ions;
SiO₂, Na₂O, CaO, P₂O₅, Al₂O₃, B₂O₃;
SiO₂, Na₂O, K₂O, CaO, MgO, P₂O₅; and/or
TiO₂, SiO₂, CaO, B₂O₃.

5. A material as defined in claim 1 which contains from 45 to 9O% of SiO₂, (typically less than 60 mol. %) and from 10 to 55% of CaO and optionally Na₂O, N₂O₅ and/or P₂O₅, especially a high sodium oxide and CaO content (20-25% each); wherein the percentages are by weight or are molar percentages; preferably the percentages are by weight.

6. A material as defined in claim 5 wherein a molar ratio of calcium to phosphorus is from 4:1 to 6:1, preferably about 5:1.

7. A material as defined in claim 1 which comprises:
60 mol. % SiO₂, 40 mol. % CaO;
70 mol. % SiO₂, 30 mol. % CaO;
60 mol. % SiO₂, 36 mol. % CaO, 4 mol. % P₂O₅;
80 mol. % SiO₂, 16 mol. % CaO, 4 mol. % P₂O₅; or
46.1 mol. % SiO₂, 24.4 mol. % Na₂O, 26.9 mol. % CaO, 2.6 mol. % P₂O₅.

8. A material as defined in claim 1 which is a bioactive ceramic, gel-glass or glass material.

9. A material as defined in claim 8 which is a bioactive ceramic material, especially a bioactive ceramic material sold under the brandname Bioglass®, more especially 45S5 Bioglass®.

10. A material as defined in claim 1 wherein the amount of bioactive material used is from 0.00001 wt %, more preferably from 0.001 wt %, most preferably from 0.01 wt % to 10 wt %, more preferably to 5 wt %, particularly preferably to 2 wt %, most preferably to 1 wt %.

11. Use of a material as defined in claim 1 in the manufacture of a medicament for use in stimulating vascularization, preferably for use in inducing secretion of an endothelial cell mitogen (especially VEGF).

12. A pharmaceutical formulation comprising a material as defined in claim 1 and a pharmaceutically acceptable adjuvant or carrier.

13. A formulation as defined in claim 12 wherein the material is included in the form of a powder.

14. A formulation as defined in claim 12 which comprises from 0.00001 wt %, more preferably from 0.001 wt %, most preferably from 0.01 wt % to 10 wt %, more preferably to 5 wt %, particularly preferably to 2 wt %, most preferably to 1 wt % of the material.

15. A formulation as defined in claim 12 for use in stimulating vascularization particularly in a wound or a burn; more preferably for use in inducing secretion of an endothelial cell mitogen (especially VEGF) particularly in a wound or a burn.

16. Use of a material as defined in claim 1 in the manufacture of a pharmaceutical formulation as defined in claim 11 for use in stimulating vascularisation, preferably for use in inducing secretion of an endothelial cell mitogen (especially VEGF).

17. A wound dressing including a dressing layer having an upper surface and a wound facing surface and a layer of bioactive material as defined in claim 1 wherein the layer of bioactive material is applied to the wound facing surface of the dressing layer.

18. A dressing as defined in claim 17 wherein the layer of bioactive material is continuous.

19. A dressing as defined in claim 17 wherein the layer of bioactive material includes a biodegradable polymer; preferably the layer is in the form of a bioactive material/biodegradable polymer composite or foam.

20. A dressing as defined in claim 17 which is in the form of a dressing which supports proliferation, preferably a polyurethane foam dressing or a hydrocolloid dressing, for example, one manufactured from pectin, gelatine, a hydrophobic polymer and/or carboxymethylcellulose.

21. A dressing as defined in claim 17 which includes a further layer which is an adhesive layer and/or a removable protective layer.

22. A method of inducing vascularization in a wound or burn which method comprises applying to a patient in need of such treatment an effective amount of a bioactive material as defined in claim 1.

23. A method as defined in claim 22 wherein the bioactive material is provided by a pharmaceutical formulation as defined in any one of claims 12 to 15 or a wound dressing as defined in claim 17.

24. A ligature including linking means for joining a first side and a second side of a wound together wherein the means is coated and/or impregnated with a bioactive material as defined in claim 1.

25. A ligature as defined in claim 24 which is in the form of a suture, surgical staple or adhesive strip.

26. A ligature as defined in claim 24 which contains a surface coating of bioactive material.

27. A ligature as defined in claim 24 wherein the amount of bioactive material used is preferably from 0.00003125 mg/cm² (0.00001 wt %), more preferably from 0.003125 mg/cm² (0.001 wt %), most preferably from 0.03125 mg/cm² (0.01 wt %) to 6.25 mg/cm² (2 wt %), preferably to 3.125 mg/cm² (1 wt %), most preferably to 1.5625 mg/cm² (0.5 wt %).

28. A tissue construct which consists essentially of a biocompatible material (preferably a biocompatible polymer), a bioactive material as defined in claim 1 and, optionally, one or more biological cells.

29. A construct as defined in claim 28 wherein the bioactive material is in the form of a powder.

30. A construct as defined in claim 28 wherein the bioactive material has an average particle size of less than about 5 μm.

31. A tissue construct which comprises a porous biocompatible material (preferably a porous biocompatible polymer) and a particulate bioactive material having an average particle size less than the average size of the pores of the polymer.

32. A construct as defined in claim 28 wherein the amount of the bioactive material used is less than 10 wt %, preferably less than 1 wt %, more preferably from 0.001 (most preferably from 0.01) to 1 wt %.

33. A tissue construct which comprises a biocompatible material (preferably a biocompatible polymer), a bioactive material as defined in claim 1 in an amount between 0.001 and 10 wt % and, optionally, one or more biological cells.

34. A construct as defined in claim 28 wherein the biocompatible material is biodegradable

35. A construct as defined in claim 34 which is biodegradable.

36. A construct as defined in claim 28 wherein the biocompatible material is a non-biodegradable biocompatible material.

37. A construct as defined in claim 28 which comprises a biocompatible polymer coated with a bioactive material or a composite of a biocompatible polymer and a bioactive material.

38. A construct as defined in claim 28 wherein the biocompatible polymer is in the form of a porous, biocompatible membrane that contains an optimal amount of bioactive material.

39. A construct as defined in claim 38 which is designed to fit into an enterocutaneous or perianal fistula.

40. A construct as defined in claim 28 which contains one or more biological cells, preferably an autologous biological cell.

41. A construct as defined in claim 40 wherein the biological cell is a fibroblast or an endothelial cell.

42. A construct as defined in claim 28 which is in the form of a hybrid organ comprising one or more biological cells from a human or animal organ.

43. A construct as defined in claim 28 for use in stimulating new tissue growth, preferably for use in stimulating vascularization, more preferably for use in inducing secretion of an endothelial cell mitogen (especially VEGF).

44. A construct as defined in claim 40 which is in the form of an intermediate dressing for use in preparing a wound bed for skin grafting.

45. A construct as defined in claim 28 for use in a high throughput screening test.

46. Use of a construct as defined in claim 28 in the manufacture of a medicament for use in stimulating new tissue growth, preferably for use in stimulating vascularization, more preferably for use in inducing secretion of an endothelial cell mitogen (especially VEGF).

47. A method of stimulating new tissue growth which method includes the step of implanting in a patient in need of such treatment an effective amount of a tissue construct as defined in claim 28.

48. A method as defined in claim 47 which is a method of stimulating vascularization, preferably a method of inducing secretion of an endothelial cell mitogen (especially VEGF).

49. A therapeutic agent delivery system which comprises a biocompatible semi-permeable membrane which encapsulates one or more biological cells and a bioactive material which is as defined in claim 1.

50. A system as defined in claim 49 wherein the therapeutic agent is an angiogenic growth factor, especially VEGF.

51. A system as defined in claim 49 wherein the membrane permits exchange of nutrients, oxygen and a biologically active product.

52. A system as defined in claim 49 wherein the membrane is a natural polymer, preferably an alginate, alginate-agarose or alginate-poly-L-lysine.
